(19) United States
(12) Patent Application Publication
(10) Pub. No.: US 2023/0223110 A1
(43) Pub. Date: Jul. 13, 2023

(54) Variant Calling For Multi-Sample Variation Graph

(71) Applicant: KONINKLIJKE PHILIPS N.V., EINDHOVEN (NL)

(72) Inventors: Zafar Ahmad, Cambridge, MA (US); Alex Ryan Mankovich, Somerville, MA (US); Yee Him Cheung, Boston, MA (US)

(21) Appl. No.: 17/761,218

(22) PCT Filed: Sep. 24, 2020
(86) PCT No.: PCT/EP2020/076785

§ 371 Date: Mar. 17, 2022

Related U.S. Application Data
(60) Provisional application No. 62/905,713, filed on Sep. 25, 2019.

(51) Int. Cl. G16B 30/20; G16B 20/20; G16B 30/10; G16H 50/30

(52) U.S. Cl. CPC G16B 30/20; G16B 20/20; G16B 30/10; G16H 50/30

(57) ABSTRACT

A method for calling variants in genetic data includes sorting nodes in a graph-based reference genome, assigning identification information to the sorted nodes, assigning depth values to respective ones of the sorted nodes, determining a reference genome path and one or more variation paths, and determining one or more variants in the graph-based reference genome based on the depth values assigned to nodes on the one or more variation paths.

### TECHNICAL FIELD

This disclosure relates generally to bioinformatics, and more specifically, but not exclusively, to processing information related to the human genome.

### BACKGROUND

Various methods have been proposed for transmuting raw genomic data. One method relies on mapping reads to a linear reference human genome using a de facto reference genome. However, a de facto reference genome represents only a tiny subset of the human population, and therefore does not credibly reflect the vast allelic diversity that exists. This results in what is referred to as allele bias, where normal (e.g., healthy) deviations from the reference genome are not represented. This results in poor read alignment accuracy for those samples which are dissimilar.

Another method proposes to transmute raw genomic data by sequencing a sample and then comparing it to a graph-based reference genome. The graph-based reference genome may incorporate more human genomes into a single structure than a de facto reference genome. However, the graph-based method also has significant drawbacks. For example, the ways in which reads are mapped to the reference graph may be different in methodology from existing methods for the linear reference genome. Moreover, the graph-based method fails to adequately compensate allele bias, which makes unsuitable for many applications.

### SUMMARY

A brief summary of various example embodiments is presented below. Some simplifications and omissions may be made in the following summary, which is intended to highlight and introduce some aspects of the various example embodiments, but not to limit the scope of the invention. Detailed descriptions of example embodiments adequate to allow those of ordinary skill in the art to make and use the inventive concepts will follow in later sections.

In accordance with one or more embodiments, a method for processing information includes sorting nodes in a graph-based reference genome; assigning identification information to the sorted nodes; assigning depth values to respective ones of the sorted nodes; determining a reference genome path and one or more variation paths; and determining one or more variants in the graph-based reference genome based on the depth values assigned to nodes on the one or more variation paths. The nodes may be topologically sorted in a predetermined direction through the graph-based reference genome.

Assigning the depth values may include assigning an initial value to a first one of the nodes, and for each subsequent one of the nodes counting a number of nodes from said each subsequent node to the first node, taking a most direct path back to the first node along the reference genome path, one or more of the variation paths, or a combination of the reference genome path and one or more of the variation paths. If the predecessor set is not empty, then a direct path may be taken to the first node. Otherwise, conditional 4 of Equation 4 (discussed below) may be taken (e.g., minimum depth value among all of its successors minus 1).

Determining the reference genome path and the one or more variation paths may include performing a global search through the nodes of the graph-based reference genome to determine the reference genome path and performing local searches for nodes along the reference genome path to determine variation paths, each of the variation paths including one or more local paths. Each of the one or more local paths connects at least one of the nodes on the reference genome path to at least one of the nodes off the reference genome path or at least two of the nodes off the reference genome path.

The one or more variants may include at least one of an insertion into the graph-based reference genome; a deletion in the graph-based reference genome; or a replacement in the graph-based reference genome. The method may also include determining a pattern based on the one or more variants, wherein the pattern corresponds to a propensity for a subject to contract a disease or guidelines for performing a clinical trial for drug approval.

In accordance with one or more other embodiments, a system for processing information includes a memory configured to store instructions and a processor configured to execute the instructions to (a) sort nodes in a graph-based reference genome, (b) assign identification information to the sorted nodes, (c) assign depth values to respective ones of the sorted nodes, (d) determine a reference genome path and one or more variation paths, and (e) determine one or more variants in the graph-based reference genome based on the depth values assigned to nodes on the one or more variation paths. The nodes may be topologically sorted in a predetermined direction through the graph-based reference genome.

The processor may assign the depth values by assigning an initial value to a first one of the nodes and for each subsequent one of the nodes counting a number of nodes from said each subsequent node to the first node, taking a most direct path back to the first node along the reference genome path, one or more of the variation paths, or a combination of the reference genome path and one or more of the variation paths.

The processor may determine the reference genome path and the one or more variation paths by performing a global search through the nodes of the graph-based reference genome to determine the reference genome path and performing local searches for nodes along the reference genome path to determine variation paths, each of the variation paths including one or more local paths. Each of the local paths may connect at least one of the nodes on the reference genome path to at least one of the nodes off the reference genome path, or at least two of the nodes off the reference genome path. The one or more variants may include at least one of an insertion into the graph-based reference genome, a deletion in the graph-based reference genome, or a replacement in the graph-based reference genome.

In accordance with one or more other embodiments, a non-transitory computer-readable medium storing instructions for causing a processor to perform operations comprising sorting nodes in a graph-based reference genome, assigning identification information to the sorted nodes, assigning depth values to respective ones of the sorted nodes, determining a reference genome path and one or more variation paths, and determining one or more variants in the graph-based reference genome based on the depth values assigned to nodes on the one or more variation paths. The nodes may be topographically sorted in a predetermined direction through the graph-based reference genome.

Assigning the depth values may include assigning an initial value to a first one of the nodes, and for each subsequent one of the nodes counting a number of nodes from said each subsequent node to the first node, taking a most direct path back to the first node along the reference genome path, one or more of the variation paths, or a combination of the reference genome path and one or more of the variation paths.

Determining the reference genome path and the one or more variation paths may include performing a global search through the nodes of the graph-based reference genome to determine the reference genome path and performing local searches for nodes along the reference genome path to determine variation paths, each of the variation paths including one or more local paths. Each of the one or more local paths may connect at least one of the nodes on the reference genome path to at least one of the nodes off the reference genome path or at least two of the nodes off the reference genome path. The one or more variants may include at least one of an insertion into the graph-based reference genome, a deletion in the graph-based reference genome, or a replacement in the graph-based reference genome. The method may also include determining a pattern based on the one or more variants, wherein the pattern corresponds to a propensity for a subject to contract a disease or guidelines for performing a clinical trial for drug approval.

### BRIEF DESCRIPTION OF THE DRAWINGS

The accompanying figures, where like reference numerals refer to identical or functionally similar elements throughout the separate views, together with the detailed description below, are incorporated in and form part of the specification, and serve to further illustrate example embodiments of concepts found in the claims and explain various principles and advantages of those embodiments.

These and other more detailed and specific features are more fully disclosed in the following specification, reference being had to the accompanying drawings, in which:

FIG. 1 illustrates an embodiment of a method for calling variants in genetic information;

FIG. 2 illustrates an example of a graph-based reference genome;

FIG. 3 illustrates an example of how depth values may be assigned to nodes in the graph;

FIG. 4 illustrates an example of a reference genome path in the graph;

FIGS. 5A to 5E illustrate examples of local searches to determine variation paths;

FIG. 6 illustrates an example of a variation path including an insertion;

FIG. 7 illustrates an example of a variation path including a deletion;

FIG. 8 illustrates an example of a variation path including a replacement; and

FIG. 9 illustrates an embodiment of a system for determining variants from genome data;

### DETAILED DESCRIPTION

It should be understood that the figures are merely schematic and are not drawn to scale. It should also be understood that the same reference numerals are used throughout the figures to indicate the same or similar parts.

The descriptions and drawings illustrate the principles of various example embodiments. It will thus be appreciated that those skilled in the art will be able to devise various arrangements that, although not explicitly described or shown herein, embody the principles of the invention and are included within its scope. Furthermore, all examples recited herein are principally intended expressly to be for pedagogical purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or (i.e., and/or), unless otherwise indicated (e.g., “or else” or “or in the alternative”). Also, the various example embodiments described herein are not necessarily mutually exclusive, as some example embodiments can be combined with one or more other example embodiments to form new example embodiments. Descriptors such as “first,” “second,” “third,” etc., are not meant to limit the order of elements discussed, are used to distinguish one element from the next, and are generally interchangeable. Values such as maximum or minimum may be predetermined and set to different values based on the application.

Example embodiments include systems and methods for performing variant calling on genetic information, which involves determining the existence and type(s) of variants on samples that have been incorporated into a graph-based genome. One or more of these embodiments include sorting nodes in a graph-based reference genome, assigning identification information to the sorted nodes, assigning depth values to respective ones of the sorted nodes, determining a reference genome path and one or more variation paths, and determining one or more variants in the graph-based reference genome based on the depth values assigned to nodes on the one or more variation paths. In at least one embodiment, the system and method may be implemented in a manner that reduces or solves the problem of allele bias present in existing methods for transmuting raw genome data. The embodiments may also be suitable for many research applications, and especially ones that require the identification of novel high-impact variants.

The system and method embodiments may achieve this improved performance by identifying one or more variants on a graph-based reference genome that has been constructed from a set (e.g., thousands or millions) of healthy and diverse human genomes incorporated into a single structure. Such a graph-based genome may represent a more complete representation of the diversity of the human genome. In one embodiment, only one type of variant of interest may be identified. In another embodiment, a plurality of variant types may be identified. The variant type(s) may include, for example, an insertion, deletion, and replacement of phenotypes in the graph-based reference genome. When considered collectively, the variants may be analyzed to spot trends and patterns that indicate the propensity of a person to develop one or more diseases (e.g., cancer, mental illness, etc.) during his or her lifetime. Determination of the variants may also be useful in managing clinical trials for purposes of gaining approval for a new drug.

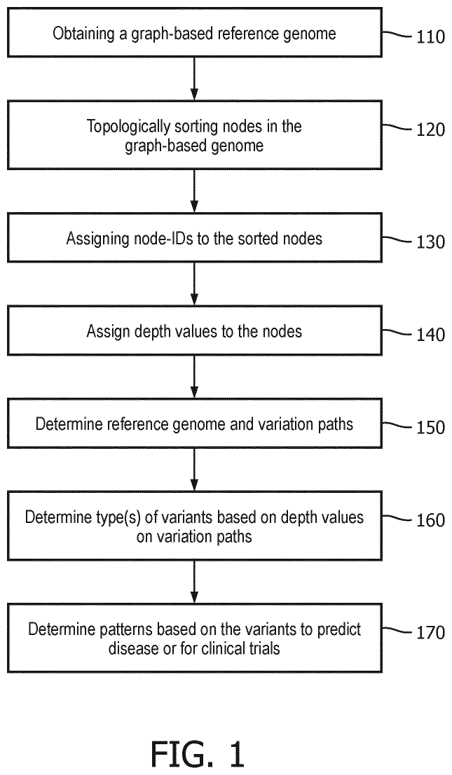

FIG. 1 illustrates an embodiment of a method for calling variants in genetic information including ones that may be found in a reference genome. While many of the embodiments are described in relation to a human genome, other embodiments may be applied to determine variants in the genome of animals. The reference genome may be generated based on thousands or millions of samples, with the latter being preferred for purposes of providing an expansive indication of genetic information representing one or more general or specific populations of interest.

At 110, the method includes obtaining the reference genome to be analyzed. The reference genome may be a graph-based human reference genome generated, for example, based on de Bruijn graph techniques, Acyclic graph techniques, Smith-Waterman techniques, or another technique or method. Such a graph includes a plurality of nodes, each corresponding to different genetic information in the genome. The graph may include edges that represent relationships between different nodes or segments in the genome. The nodes and paths may be analyzed to call variants in accordance with the embodiments herein.

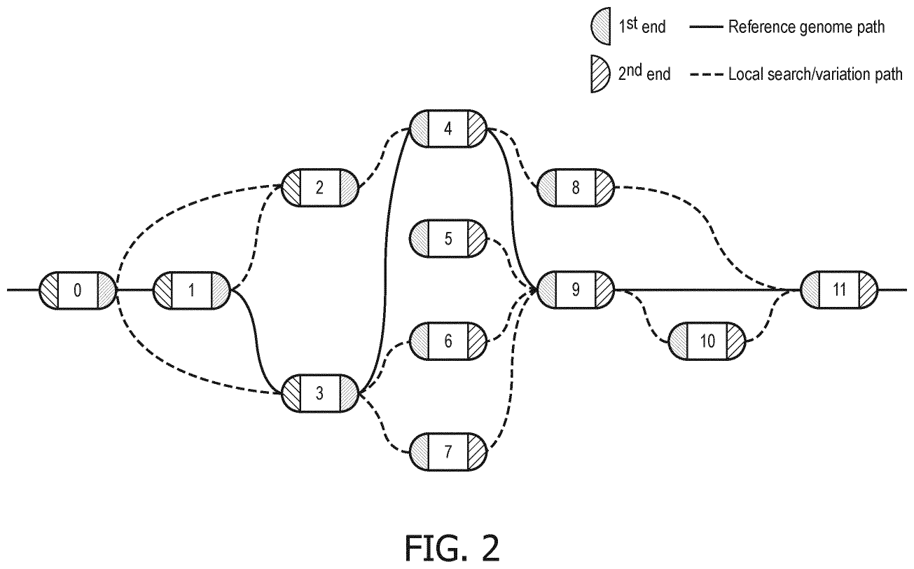

FIG. 2 illustrates an example of a graph-based reference genome which includes twelve nodes numbered 0 to 11. While only twelve nodes are illustrated in the graph of FIG. 1, it is understood that the system and method embodiments may apply to a graph having a different number of nodes, e.g., less or greater than twelve. In one embodiment, the graph may have hundreds or thousands of nodes or the system and method embodiments may only be focused locally on a reduced number of nodes in such a graph.

Nodes 0 to 11 in the reference genome may correspond to (or be indicative of) a respective number of phenotypes and are connected by at least one of two types of paths. The first type of path is a reference genome path indicating a connection of nodes (or phenotypes) representative of a reference population. The nodes connected along the reference genome path are considered to correspond to those phenotypes of subjects in a general or predetermined population. For example, the population may include or consist of what is regarded as normal and healthy population by medical or biological standards. In another implementation, the population may include or consist of subjects having a specific collection of genetic traits and/or other features of interest, whether considered normal or not.

The second type of path is considered to be a variant path and may correspond to all paths not included in the reference genome path. Each variant path may graphically appear as connected, directly or indirectly, to at least one node along the reference genome path. The connection may involve the variant path shooting off from a node on the reference genome path, coming into a node on the reference genome path from another node (on the reference path or a variant path), or connected between two nodes on the reference genome path. As will be described in greater detail below, each node on the variant path may also be connected to multiple nodes of the aforementioned types. In terms of traversing the graph, the reference genome and variant paths may be bidirectional or unidirectional, or a combination of the two at different portions of the graph. For illustrative purposes, all the paths in the graph will be discussed as a bidirectional, traversing from left to right and also right to left through the twelve nodes. As such, each node in the graph-based reference genome may include two ends in respective sides of the node.

After the graph-based reference genome is acquired, the system and method embodiments may be implemented to label variants in the graph. The variants to be labeled may be a predetermined type or multiple types. Examples of variants that may be labeled (or called) in the graph include, but are not limited to, insertions, deletions, and replacements, as described in greater detail below.

In order to identify the type of variant, the system and method may be applied to solve the following problem: Given a multi-sample variation graph G=(V, E), which includes a finite set of nodes V={v₁, v₂, . . . , vₙ} and a set of edges E⊆V×V, extract the variations with respect to the reference genome path. In this example graph of FIG. 2, n is equal to 12 corresponding to the twelve nodes. In terms of the edges E, in graph theory the degree of a node of a graph is the number of edges that are incident to the node. If there are no variations starting from a current node, the following Lemma may be satisfied: If an inner node (e.g., other than a start node and an end node on the reference genome path) has a maximum degree ≤2, that node may not serve as a start index or end index of a variation path. This may be explained as follows.

In FIG. 2, graph G is constructed to have linear connectivity along the reference genome path, and the nodes arranged along the reference genome path may not be considered to have any variation. In one embodiment, the nodes along the reference genome path may be defined as follows. An inner node may be one connected with a previous node and a next node. Each inner node may therefore have a maximum degree of 2. For each of the start node and the end node of the reference genome path, the degree may be 1. The start node is node 0 and the end node is node 11 in FIG. 2. Given this understanding, the method may continue as follows.

At 120, a sorting operation is performed for nodes in graph G. In one embodiment, all the nodes in the graph G may be sorted topologically (e.g., start node, inner nodes, and end node) relative to the reference genome path. The sorting may be performed in a predetermined direction. If the reference genome graph is bidirectional, then the sorting operation may be performed in a forward direction or a reverse direction. If the reference genome graph is unidirectional, then the sorting operation may be performed in the only valid direction of the graph. An example of the sorting operation is illustrated FIG. 2.

At 130, an identification number (node-id) is assigned (or re-assigned) to each node such that, traversing the graph in a predetermined direction, all predecessor nodes relative to a given node i has a node-id less than i and all successor nodes relative to node i has node-id greater than i. This operation may be performed for the start node, inner nodes, and the end node. The result is to assign node-ids in ascending order to the nodes in the graph in the predetermined direction. An example of the node assignment operation is illustrated FIG. 2.

At 140, once the nodes have been topologically sorted and node-ids have been assigned (or re-assigned) to the nodes in the graph G, depth values may be assigned to each of the nodes. This may be accomplished as follows. Assume that the following Equations (1) to (3) are true:

R={r|r∈V and r∈reference path} (1)

Pᵢ={p|p∈V and p is a predecessor of node i} (2)

Sᵢ={s|s∈V and s is a successor of node i} (3)

In the above equations, V corresponds to a finite set of nodes, R corresponds to the set of nodes that lies in reference path, Pᵢ corresponds to the set of nodes that are predecessor of node i, Sᵢ corresponds to the set of nodes that are successor of node i, and r, p, and s are variables. Based on these assumptions, depth values may be assigned to each node of graph G based on the set of Equations (4).

$$depth ( i ) = \{ \begin{matrix}1 , & if i ∈ R and i is the first node of reference path \\ {min}_{j} depth ( j ) + 1 , & if j < i and j ∈ R and j ∈ {P}_{i} \\ {min}_{k} depth ( k ) + 1 , & if k < i and k ∈ {P}_{i} \\ {max}_{l} depth ( l ) - 1 , & if l > i and {P}_{i} = ∅ and l ∈ S\end{matrix} \qquad ( 4 )$$

In Equations (4), i indicates the node identification number (node-id) and j and l are variables. The expression

$${min}_{j} depth ( j )$$

means to minimize over all nodes j that are part of the node set V. The expression

$${min}_{k} depth ( k )$$

means to minimize over nodes k. The expression

$${max}_{l} depth ( l )$$

means to maximize over nodes l. The expressions j<l, k<I, and l>I are constraints on the aforementioned equations. For example, j<l means all nodes in set V with the node id less than i.

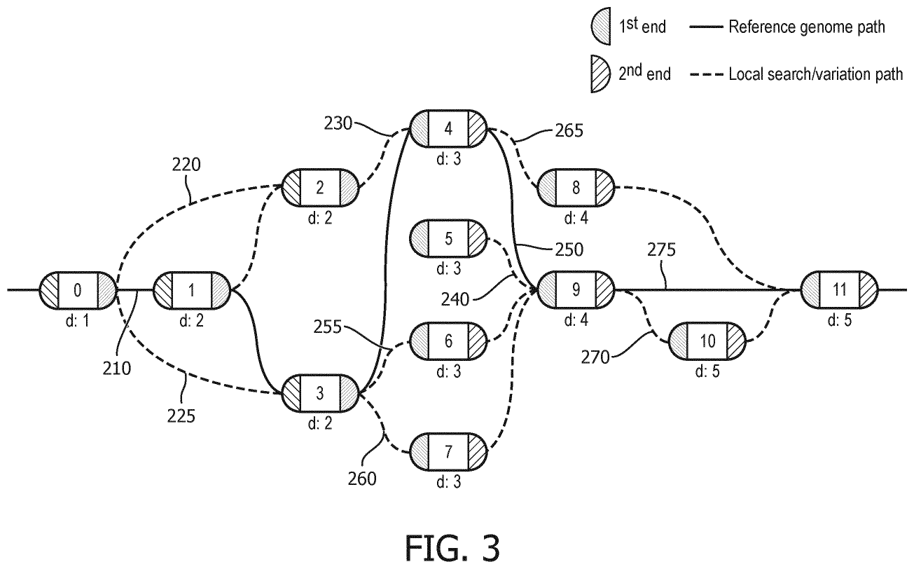

FIG. 3 illustrates an example of how depth values (d) may be assigned to the nodes 0 to 11 in FIG. 1 using the rules set forth in the set of Equations (4). The depth values calculated and assigned to the nodes may also be understood as count values in the sequence of nodes, taking the most direct path possible back to the start node—whether the most direct path is along the reference genome path only, along one or more variation paths only, or a combination of segments of the reference genome path and one or more variation paths. Put differently, the depth value may be the number of nodes removed from the start node, with the start node in the genome reference path being assigned as the first node. Thus, the nodes in the graph G of FIG. 3 may be assigned as follows.

The first node (i=node-id=0) is assigned a depth value of 1 (d:1) because this node is the first node (start node) in the sequence of nodes (count value=1) arranged along the reference genome path in the graph G.

The second node (i=node-id=1) is assigned a depth value of 2 (d:2) because this node is a second node in the sequence of nodes (count value=2) from the start node, taking the most direct path back to the start node. In this case, the most direct path back to the start node is path 210 which is situated along the reference genome path.

The third node (i=node-id=2) is assigned a depth value of 2 (d:2) because this node is another second node in the sequence of nodes (count value=2) from the start node, taking the most direct path back to the start node. In this case, the most direct path back to the start node is through variation path 220 (which bypasses the second node 1).

The fourth node (i=node-id=3) is assigned a depth value of 2 (d:2) because this node is another second node in the sequence of nodes (count value=2) from the start node, taking the most direct path back to the start node. In this case, the most direct route back to the start node is through variation path 225.

The fifth node (i=node-id=4) is assigned a depth value of 3 (d:3) because this node is a third node in the sequence of nodes (count value=3) from the start node, taking the most direct path back to the start node. In this case, the most direct route back to the start node is through variation paths 220 and 230. This route passes through inner node 2.

The sixth node (i=node-id=5) is assigned a depth value of 3 (d:3) because this node doesn't have a direct path via its predecessor (it's predecessor set is empty) to the start node. Based on equation 4, conditional 4, the depth value of this node must be calculated from its successor via path 240. Its success is node 9 and node 9 has depth value of 4. Therefore, it's depth value is 3.

The seventh node (i=node-id=6) is assigned a depth value of 3 (d:3) because this node is a third node in the sequence of nodes (count value=3) from the start node, taking the most direct path back to the start node. In this case, the most direct route back to the start node is through variation paths 225 and 255. This route passes through inner node 3.

The eighth node (i=node-id=7) is assigned a depth value of 3 (d:3) because this node is a third node in the sequence of nodes (count value=3) from the start node, taking the most direct path back to the start node. In this case, the most direct route back to the start node is through variation paths 220 and 260. This route passes through inner node 3.

The ninth node (i=node-id=8) is assigned a depth value of 4 (d:4) because this node is a fourth node in the sequence of nodes (count value=4) from the start node, taking the most direct path back to the start node. In this case, the most direct route back to the start node is through variation paths 220, 230, and 265. This route passes through inner node 2 and inner node 4.

The tenth node (i=node-id=9) is assigned a depth value of 4 (d:4) because this node is a fourth node in the sequence of nodes (count value=4) from the start node, taking the most direct path back to the start node. In this case, the most direct route back to the start node is through the portion 250 of the reference genome path and variation paths 220 and 230. This route passes through inner node 2 and inner node 4.

The eleventh node (i=node-id=10) is assigned a depth value of 5 (d:5) because this node is a fifth node in the sequence of nodes (count value=5) from the start node, taking the most direct path back to the start node. In this case, the most direct route back to the start node is through variation paths 220, 230, and 270 and the portion 250 of the reference genome path. This route passes through inner node 9, inner node 4, and inner node 2.

The twelfth node (i=node-id=11) is the end node and is assigned a depth value of 5 (d:5) because this node is a fifth node in the sequence of nodes (count value=5) from the start node, taking the most direct path back to the start node. In this case, the most direct route back to the start node is through variation paths 220 and 230 and portions of the reference genome path 250 and 275. This route passes through inner node 2, inner node 4, and inner node 9. (Other routes exist that will also produce this depth value for node 11).

Using this approach and the set of Equations (4), depth values for the nodes in the graph-based genome may be calculated and assigned as indicated in Table 1.

TABLE 1

| i (node id) | Pᵢ | Sᵢ | Conditional | depth(i) |
|---|---|---|---|---|
| 0 | Ø | {1, 2, 3} | 1 | 1 |
| 1 | {0} | {2, 3} | 2 | 2 |
| 2 | {0, 1} | {4} | 2 | 2 |
| 3 | {0, 1} | {4, 5, 6, 7} | 2 | 2 |
| 4 | {2, 3} | {8, 9} | 2 | 3 |
| 5 | Ø | {9} | 4 | 3 |
| 6 | {3} | {9} | 3 | 3 |
| 7 | {3} | {9} | 3 | 3 |
| 8 | {4} | {11} | 2 | 4 |
| 9 | {4, 5, 6, 7} | {10, 11} | 2 | 4 |
| 10 | {9} | {11} | 2 | 5 |
| 11 | {8, 9, 10} | Ø | 2 | 5 |

At 150, the reference genome path and the variation paths in the graph-based reference genome may be determined using a two-stage recurrent search. In one embodiment, operation 150 may be performed after operation 140 that assigns depth values to the nodes in the graph. In another embodiment, operation 150 may be performed concurrently with operation 140.

In performing the two-stage recurrent search, the first type of search may be performed to locate a global search path in the graph, which corresponds to the reference genome path. This may be explained as follows. Initially, the graph construction starts with a reference genome and then a number of iterations are performed. In each iteration of a different reference genome sample alignment to the graph, variations are added to the graph. Therefore, the reference genome graph gets enriched in each iteration. While adding these reference nodes, a marker is assigned to the nodes so that it can be identified as the part of the reference genome in future.

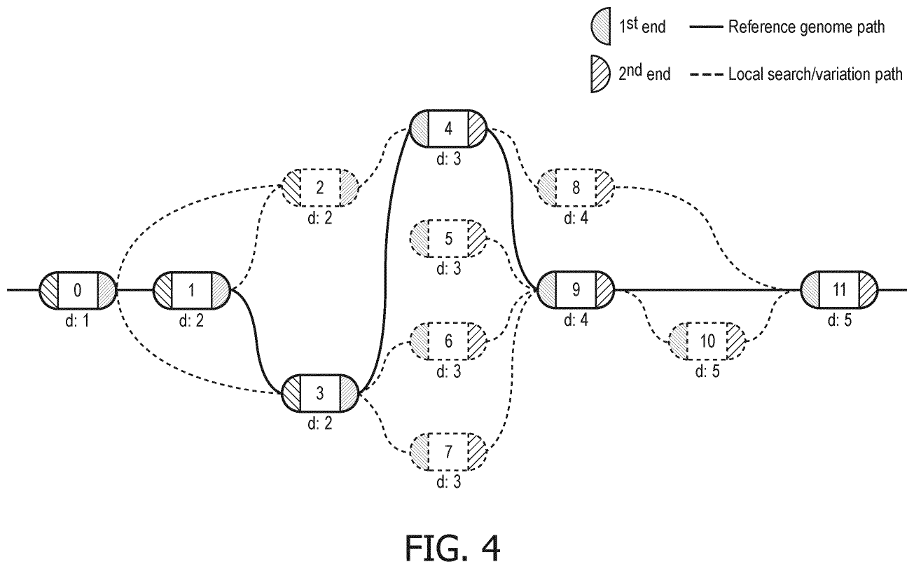

In the graph of FIG. 3, the reference genome path is the path connecting nodes 0→1→3→4→9→11, as emphasized in FIG. 4. For clarification, the reference genome (or global search) path produced by the global search is the isolated path traversing the aforementioned nodes. Global and local searches may not be disjoint operations. For example, a global search is performed by traversing along the reference genome path. If the max-degree of a node >2 (e.g., for start and end node of reference path, max-degree >1) is encountered, a local search may be launched.

Once the global search has been performed to determine the reference genome path, the second type of search of the two-stage recurrent search may performed. The second type of search is performed to determine variation paths in the graph. In performing this search, it is to be understood that multiple variation paths may traverse through or be associated with a single node, situated either along the reference genome path or a variation path. Thus, each path may be extracted in the second-stage search may be considered as a valid variation.

In performing this search, traversing the graph from left to right, when a maximum degree (max-degree) of any inner node >2 (or max-degree >1 for the start node and the end node on the reference genome path) is found during the global search, a local search may then be performed. Because graph G has been topologically sorted and node-ids have been assigned (e.g., in increasing order), a node may be visited if it has a node-id strictly greater than the current node i (using re-assignment of node ids previously performed). When a node is discovered which is along the reference genome path, the local search may be stopped and the associated traverse path(s) may be added as a variation(s). Thus, multiple local searches may be performed for at least some of the nodes.

As previously indicated, operations 140 and 150 may be performed concurrently. In this case, after the reference genome path is determined, the graph is traversed in a predetermined search direction (e.g., from left to right) beginning with the first node along the reference genome path, which is start node 0. At this time, the depth value for start node 0 is calculated. Then, a local search is performed relative to node 0 to determine variation path(s) stemming from node 0. Depth values for the nodes along those variation path(s) are then calculated. Subsequently, the depth value for the next node along the reference genome path is calculated. A local search is then performed relative to the next node to determine variation path(s) stemming from that node. Depth values for the nodes along those variation path(s) are then calculated. This process continues for subsequent nodes along the reference genome path until depth values are calculated for all nodes and all variation paths in the graph have been determined.

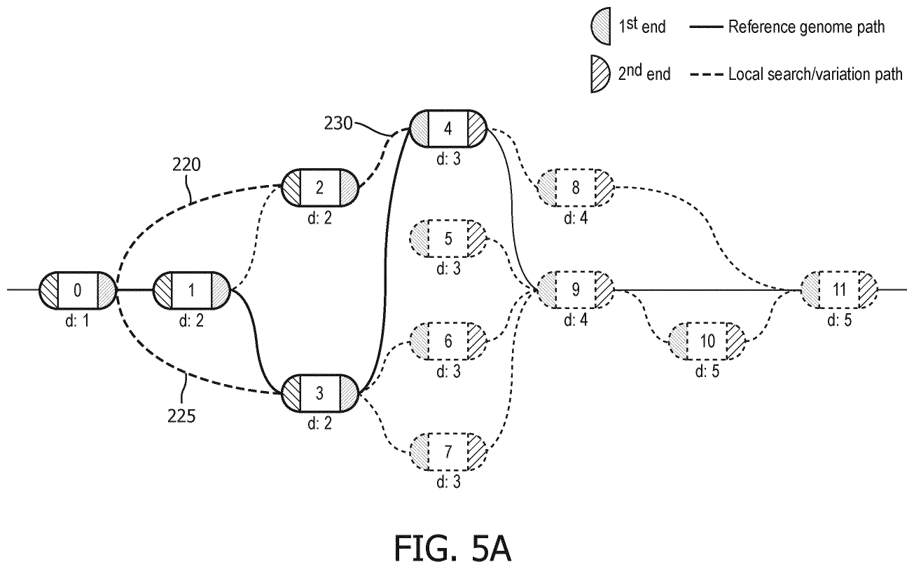

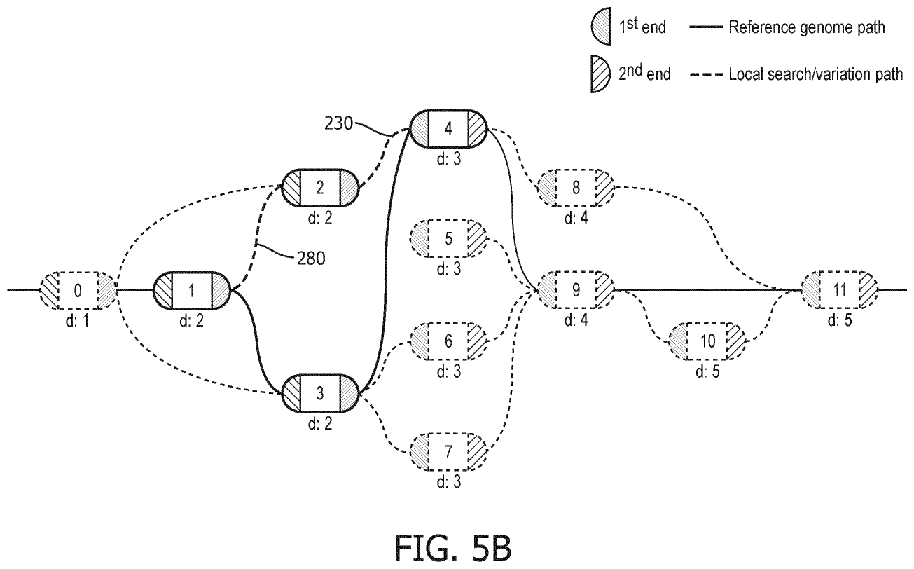

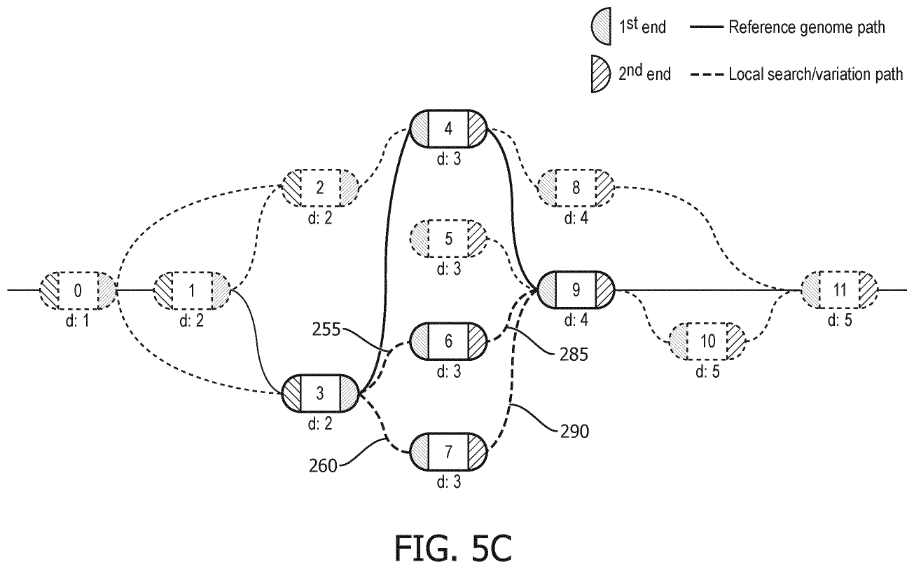

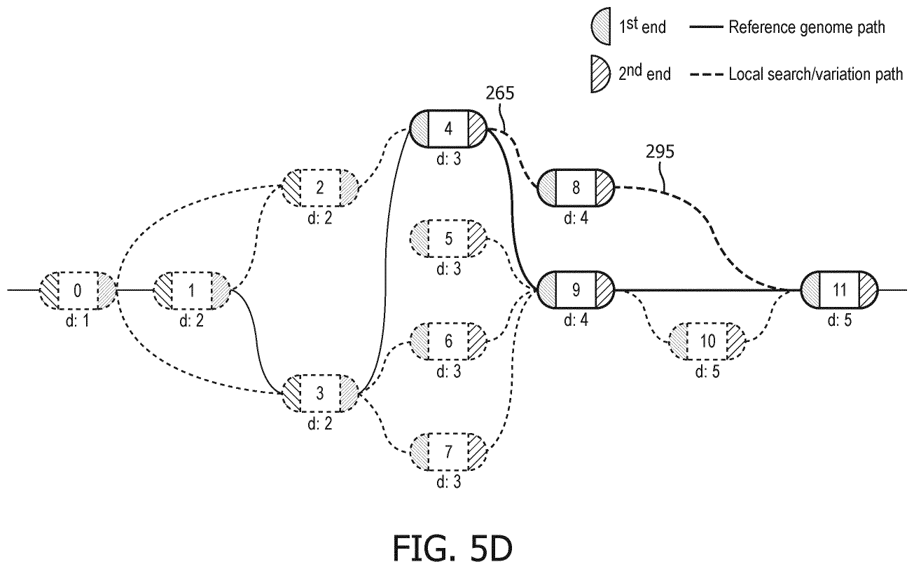

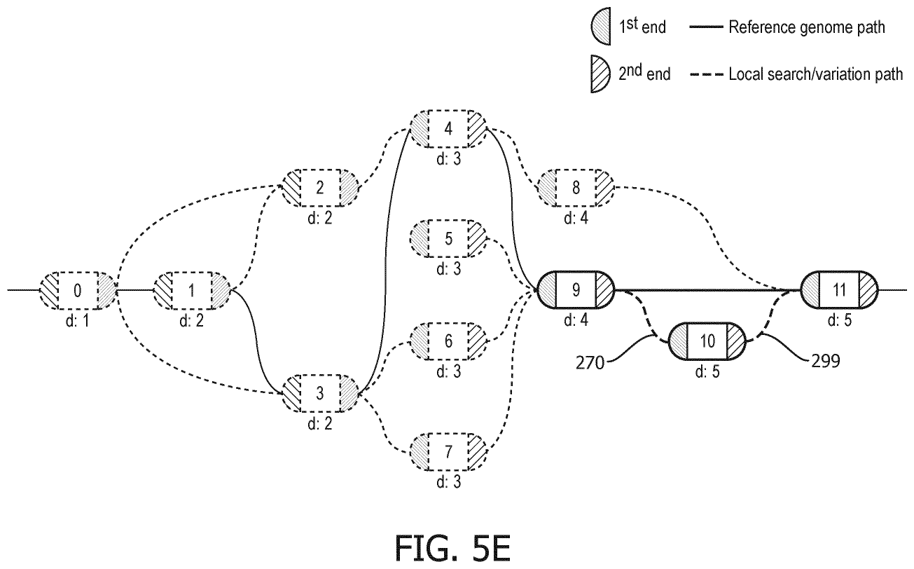

FIGS. 5A to 5E illustrate examples of local searches that may be performed to determine variation paths in the graph-based reference genome. In FIGS. 5A to 5E, these local searches are performed after depth values for all nodes in the graph have been calculated are integrated with performance of the global search. During the global search, if the depth value of an inner node along the reference genome (or global) path is >2 relative to the start node (or end node, if traversing through the graph in the reverse direction), then a local search is initiated for that inner node. If the inner node is considered relative to a node which is not the start node or the end node, then a local search is performed if the degree of a node >1.

Referring to FIG. 5A, node 4 was determined to have a depth value of 3 during the global path search. Thus, the pre-condition for performing the local search for node 4 has been satisfied. Accordingly, a local search is performed from node 0 (start node) and involves traversing the local paths from this node. The local search is terminated when a node is identified as being in the global path. In the present case, two variation paths are found by the local search. The first variation path starts at node 0 and traverses through node 2 to node 4 along local paths 220 and local path 230, respectively. The first variation path may be expressed as 0→2→4. The second variation path starts a node 0 and passes to node 3 along local path 225. The second variation path may be expressed as 0→3. In this example the variants along the two local paths correspond to different types, e.g., the first variation path is determined as corresponding to a replacement or structural variation and the second variation path is determined as corresponding to a deletion.

Referring to FIG. 5B, a local search may also be performed beginning with node 1. Such a search will identify a variation path including nodes 1, 2, and 4 along local paths 230 and 280. This variation path may be expressed as 1→2→4. Though the depth value difference between the beginning node (node 1) and end node (node 4) is 1, node 3 has a duplicate depth value 2 which lies between the beginning and end nodes (1 and 4). As explained in greater detail below, the variation path 1→2→4 may be classified as a replacement or structural variation.

Referring to FIG. 5C, a local search may also be performed beginning with node 3. Such a search determines a first variation path including nodes 3, 6, and 9 along local paths 255 and 285 and a second variation path including nodes 3, 7, and 9 along local paths 260 and 290. In both the cases, the difference between depth values of the beginning node (node 3) and end node (node 9) is >1. As discussed in greater detail below, both variations paths in this case may be classified as the same type of variant, namely a replacement or structural variation.

Referring to FIG. 5D, a local search may also be performed beginning with node 4. Such a search determines a variation path including nodes 4, 8, and 11 along local paths 265 and 295. This variation path may be expressed as 4→8→11. As will be discussed in greater detail below, the variant associated with this path is a replacement or structural variation.

Referring to FIG. 5E, a local search may also be performed beginning with node 9. Such a search determines a variation path including nodes 9, 10, and 11 along local paths 270 and 299. This variation path may be expressed as 9→10→11. The difference between the depth value of the beginning node (node 9) and the end node (node 11) is ≤1. Therefore, there is no node with the duplicate depth value in the global search path between this beginning and an end node. As discussed in greater detail below, the variant associated with variation path 9→10→11 is an insertion variation.

At 160, once the depth values have been assigned and the variation paths determined, the types of variants corresponding to the nodes that are connected to nodes along the reference genome path may be determined. The variant nodes may include the ones situated along one or more of the variation paths previously indicated. In one embodiment, the graph may include only one type of variant. In other embodiments, the graph may include multiple types of variants. In the example of FIG. 3, the graph includes three types of variants: insertions, deletions, and replacements.

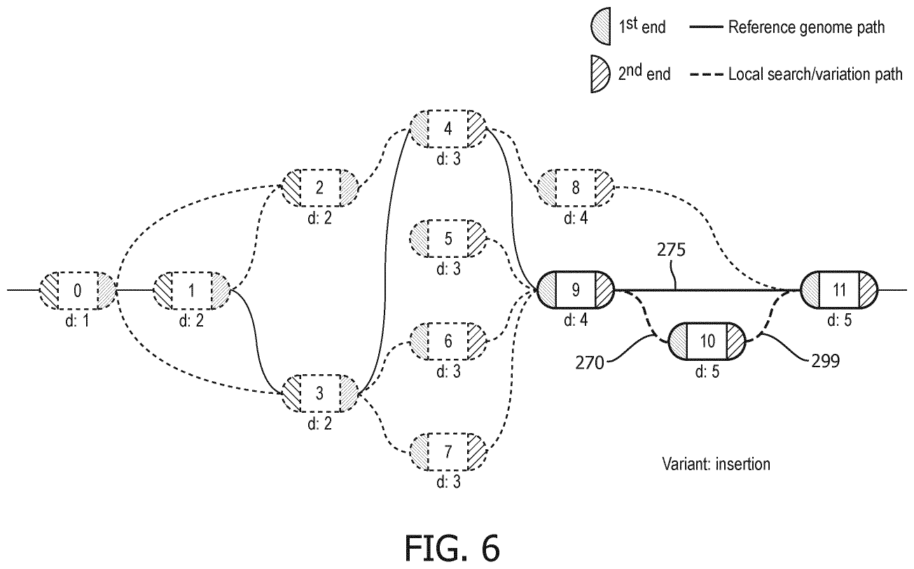

FIG. 6 illustrates an example where variant node 10 has been inserted into (e.g., as an off-shoot or deviation from) the reference genome path between nodes 9 and 11. In one embodiment, determining the existence of an insertion in the graph-based human genome G may involve an examination of adjacent nodes located along the reference genome path.

More specifically, a search may be performed to locate adjacent nodes along the reference genome path which are connected to a node that is not on the reference genome path, and the node that is not on the genome path is connected to the adjacent nodes by one or more variation paths, which also may be referred to as local search paths. When such a pair of adjacent nodes is found, an insertion of a node may be determined to exist when the difference in depth values between the adjacent nodes is ≤1. In this case, the local search path includes one or more nodes between the adjacent nodes, but the global search path does not include any node with a duplicate depth value between the adjacent nodes. In this case, the variation may be described as an insertion corresponding to the node on the local search path.

Such a case is illustrated in FIG. 6, where node 9 (node-id=9) and node 11 (node-id=11) are adjacent to one another along the portion 275 of the reference genome path and node 10 is between nodes 9 and 11 along the variation path which includes local search paths 270 and 299 connecting the adjacent nodes. There are no nodes with duplicate depth values between node 9 and node 11. The local search path may be expressed as 9→10→11. In determining whether node 10 constitutes an insertion, the depth values of node 9 and node 11 must be determined. Node 9 has been assigned a depth value of 4 (d:4) and node 11 has been assigned a depth value of 5 (d:5). Thus, the difference between the depth values of node 9 and node 11 is therefore ≤1. Thus, it may be determined that node 10 (e.g., the contents of node 10, which may be a phenotype) is an inserted variant or sequence.

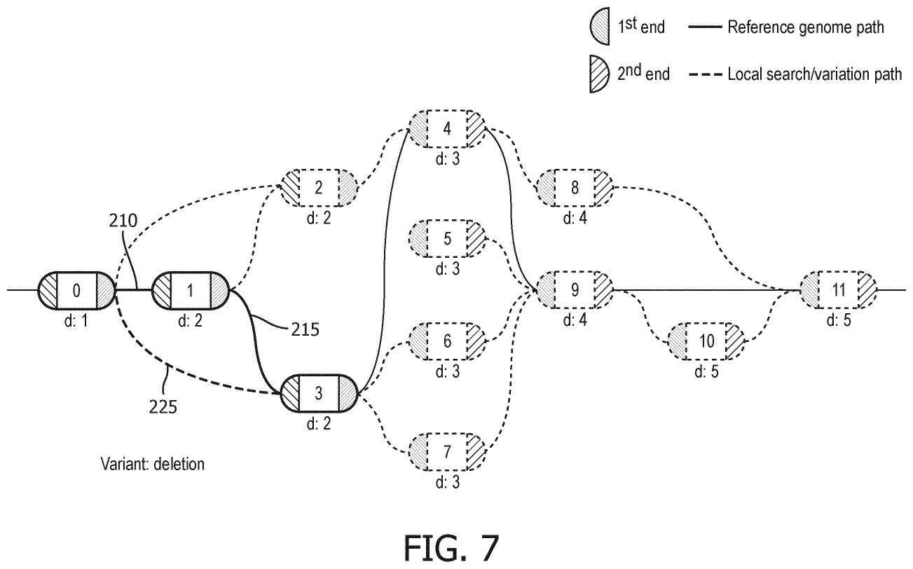

FIG. 7 illustrates an example where a node is determined to be deleted along a variation (or local search) path connecting nodes on the reference genome path. The deleted node may be an inner node between nodes which is also on the reference genome path.

More specifically, determining the existence of a deletion in the graph-based human genome G may initially involve an examination of the nodes along the reference genome path. When an inner node is located between two other nodes on the reference genome path, a determination is made as to whether a variation (or local search) path exists that connects the two other nodes. The two other nodes may themselves be inner nodes or one of the two other nodes may be a start node or an end node in the graph G. If the depth value of the inner node is equal to the depth value of one of the other two nodes, then the inner node may be determined to have been deleted from the variation (or local search) path connecting the other two nodes.

Referring to FIG. 7, applying these operations, inner node 1 is determined to be located between node 0 and node 3, all of which are located along the reference genome path, and specifically portions 210 and 215 of the reference genome path. Additionally, a variation or local search path 225 is determined to connect node 0 and node 3. Now, the depth values are examined. Node 0 has a depth value of 1, node 1 has a depth value of 2, and node 3 has a depth value of 2. Because inner node 1 has a depth value that is equal to the depth value of one of the nodes 1 or 3 (in this case, node 1 and node 3 have the same depth value), it may be determined that there has been a deletion of node 1 along the variation path 220 connecting node 1 and node 3.

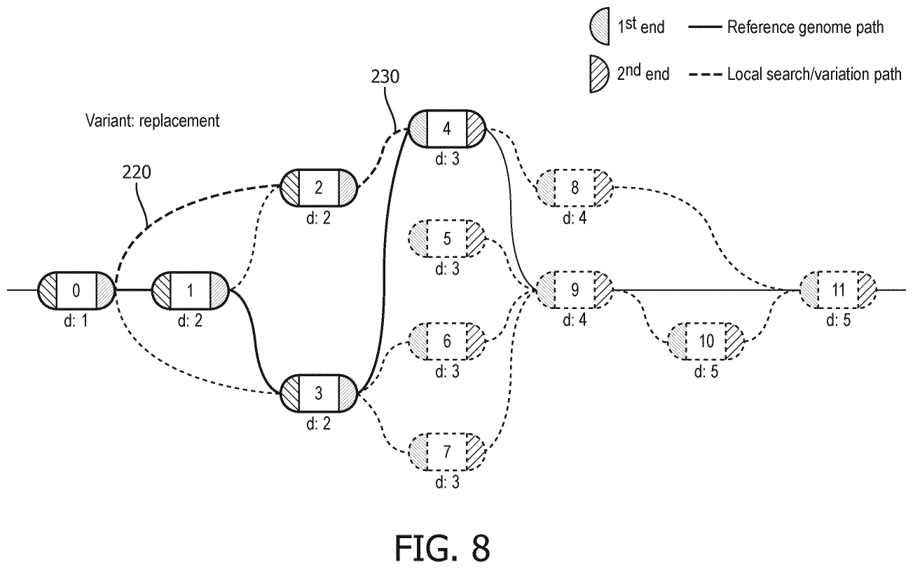

FIG. 8 illustrates an example of where a replacement (or structural variation) has occurred in the graph-based genome G. A replacement or structural variation of a node may correspond to all those nodes that are along variation or local search paths (and thus not along the reference genome path) that have not been determined to be an insertion or deletion. For example, if the length of a variation path is a single nucleotide, then the node(s) along that path may be determined to be a single nucleotide replacement. Thus, for example, node 2 in FIG. 8 connected to the variation path which includes local search paths 220 and 230 (e.g., 0→2→4) may be determined to be a replacement of node 1 and node 3 along the reference genome path. Applying these principles, node 8 may be determined to be a replacement for node 9 and each of node 6 and node 7 may be determined to be a replacement for node 4.

In FIG. 1, at 170, the variants may be processed to determine the existence of patterns that may be used as a basis for performing various applications. For example, patterns of the variants may be correlated to certain diseases or a propensity for a subject to develop a certain disease later on in life. The embodiments disclose herein may therefore be used as an early warning detector that may cause subjects to modify their lifestyles in order to live to an older age. In another example, patterns of the variants may be used as a basis for developing guidelines or subject selection during clinical trials during the approval process for a new drug.

According to another example, genomic variants may be used in a variety of clinical applications. For example, in germline testing, a practical application of the embodiments described herein is for variants associated with a predisposition for cancers, such as BRCA1/BRCA2 variants for breast cancer and TP53 variants for a variety of cancer types. Variants identified in particular cancers may be used as therapeutic targets, e.g., non-small cell lung cancer patients with the BRAF V600E mutation may benefit from Dabrafenib.

In one practical application, a collection of variants that tend to occur together (e.g., are co-inherited) may be termed a haplotype. In some cases, haplotypes are associated with particular conditions or disease susceptibility. There are several such examples for complex diseases like schizophrenia, where disease risk has been associated with haplotypes in DLG4, COMT, and other genes.

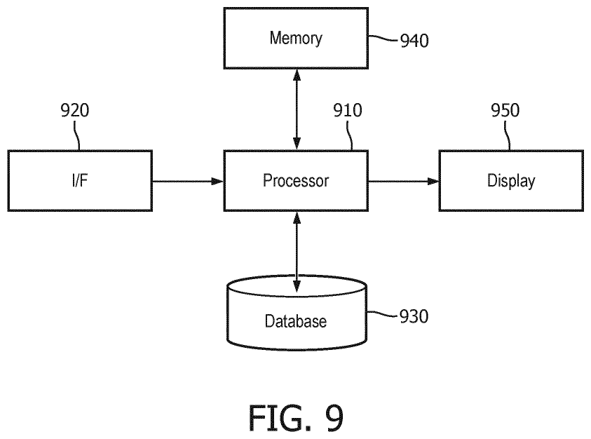

FIG. 9 illustrates an embodiment of a system for determining variants from genome data. The system includes a processor 910, an interface 920, a database 930, a memory 940, and a display 950. The processor may by a computer, workstation, server, or other processing or computing device. The processor may receive genome data through the interface 920 and store this data in database 930. This data may be received in raw form, in which case the processor 910 may process the data to generate the graph-based reference genome as previously discussed. In one embodiment, the data may already be in graph form. In this case, the processor 910 may store the data in the database without substantial processing concerning the graphical format of the data.

The memory 940 may store instructions to be executed by the processor for performing the operations included in the method embodiments described herein. By executing these instructions, the processor 910 may determine the existence of variants in the received genome data and the types of those variants, as previously described. The processor 910 may execute additional instructions to locate trends and/or patterns that may be used as a basis for predicting, for example, whether an individual having one or more of the variants is likely to develop a disease or other condition during his or her lifetime. The embodiments may also be applied to perform other applications, a non-limiting example of which is discussed in greater detail below. A visual representation of the variants, global (reference genome) path, local (variation) paths, nodes, and other data may be output from the processor 910 to the display 950.

In accordance with one embodiment, a non-transitory computer-readable medium may store instructions which, when executed by a processor, performs the operations of the method embodiments described herein. The computer-readable medium may be a read-only memory, random-access memory, flash memory, or another type of memory. In one embodiment, the computer-readable medium may correspond to memory 740 in FIG. 7 for causing processor 710 to perform the operations of the embodiments described herein.

### Example Applications

An example application of the embodiments relates to the performance of cohort studies. In such an application, a pharmaceutical company developing a line of immunotherapy drugs would like to identify novel markers that can predict response to therapy. Researchers assemble a large graph genome comprised of 5,000 tumor sequences pulled from patients that underwent immunotherapy. Each sequence is associated with a patient, along with clinical data for that patient, including demographics and therapy response. Using the embodiments described herein, variants are determined across the HLA region (e.g., a key region associated with immune response in humans) of the graph for each patient. The resulting data may be analyzed or processed to identify a strong association of a particular haplotype, where certain variants were co-inherited across the HLA-A, HLA-B, and HLA-C genes, with a positive response to a variety of immunotherapies. The pharmaceutical company may then use this knowledge as selection criteria for clinical trials to be performed for the new line of immunotherapy drugs.

Although the various exemplary embodiments have been described in detail with particular reference to certain exemplary aspects thereof, it should be understood that the invention is capable of other example embodiments and its details are capable of modifications in various obvious respects. As is readily apparent to those skilled in the art, variations and modifications can be affected while remaining within the spirit and scope of the invention. Accordingly, the foregoing disclosure, description, and figures are for illustrative purposes only and do not in any way limit the invention, which is defined only by the claims.

1. A method for processing information, comprising:
sorting nodes in a graph-based reference genome;
assigning identification information to the sorted nodes;
assigning depth values to respective ones of the sorted nodes;
determining a reference genome path and one or more variation paths; and
determining one or more variants in the graph-based reference genome based on the depth values assigned to nodes on the one or more variation paths.

2. The method of claim 1, wherein the nodes are topographically sorted in a predetermined direction through the graph-based reference genome.

3. The method of claim 1, wherein assigning the depth values includes:
assigning an initial value to a first one of the nodes, and
for each subsequent one of the nodes, counting a number of nodes from said each subsequent node to the first node, taking a most direct path back to the first node along the reference genome path, one or more of the variation paths, or a combination of the reference genome path and one or more of the variation paths.

4. The method of claim 1, wherein determining the reference genome path and the one or more variation paths includes:
performing a global search through the nodes of the graph-based reference genome to determine the reference genome path; and
performing local searches for nodes along the reference genome path to determine variation paths, each of the variation paths including one or more local paths.

5. The method of claim 4, wherein each of the one or more local paths connects:
at least one of the nodes on the reference genome path to at least one of the nodes off the reference genome path, or
at least two of the nodes off the reference genome path.

6. The method of claim 1, wherein the one or more variants include at least one of:
an insertion into the graph-based reference genome;
a deletion in the graph-based reference genome; or
a replacement in the graph-based reference genome.

7. The method of claim 1, further comprising:
determining a pattern based on the one or more variants,
wherein the pattern corresponds to a propensity for a subject to contract a disease or guidelines for performing a clinical trial for drug approval.

8. A system for processing information, comprising:
a memory configured to store instructions; and
a processor configured to execute the instructions to:
sort nodes in a graph-based reference genome;
assign identification information to the sorted nodes;
assign depth values to respective ones of the sorted nodes;
determine a reference genome path and one or more variation paths; and
determine one or more variants in the graph-based reference genome based on the depth values assigned to nodes on the one or more variation paths.

9. The system of claim 8, wherein the nodes are topographically sorted in a predetermined direction through the graph-based reference genome.

10. The system of claim 8, wherein the processor is to assign the depth values by:
assigning an initial value to a first one of the nodes; and
for each subsequent one of the nodes counting a number of nodes from said each subsequent node to the first node, taking a most direct path back to the first node along the reference genome path, one or more of the variation paths, or a combination of the reference genome path and one or more of the variation paths.

11. The system of claim 8, wherein the processor is configured to determine the reference genome path and the one or more variation paths by:
performing a global search through the nodes of the graph-based reference genome to determine the reference genome path; and
performing local searches for nodes along the reference genome path to determine variation paths, each of the variation paths including one or more local paths.

12. The system of claim 11, wherein each of the one or more local paths connects:
at least one of the nodes on the reference genome path to at least one of the nodes off the reference genome path, or
at least two of the nodes off the reference genome path.

13. The system of claim 8, wherein the one or more variants include at least one of:
an insertion into the graph-based reference genome;
a deletion in the graph-based reference genome; or
a replacement in the graph-based reference genome.

14. A non-transitory computer-readable medium storing instructions for causing a processor to perform operations comprising:
sorting nodes in a graph-based reference genome;
assigning identification information to the sorted nodes;
assigning depth values to respective ones of the sorted nodes;
determining a reference genome path and one or more variation paths; and
determining one or more variants in the graph-based reference genome based on the depth values assigned to nodes on the one or more variation paths.

15. The computer-readable medium of claim 14, wherein the nodes are topographically sorted in a predetermined direction through the graph-based reference genome.

16. The computer-readable medium of claim 14, wherein assigning the depth values includes:
assigning an initial value to a first one of the nodes, and
for each subsequent one of the nodes, counting a number of nodes from said each subsequent node to the first node, taking a most direct path back to the first node along the reference genome path, one or more of the variation paths, or a combination of the reference genome path and one or more of the variation paths.

17. The computer-readable medium of claim 14, wherein determining the reference genome path and the one or more variation paths includes:
performing a global search through the nodes of the graph-based reference genome to determine the reference genome path; and
performing local searches for nodes along the reference genome path to determine variation paths, each of the variation paths including one or more local paths.

18. The computer-readable medium of claim 17, wherein each of the one or more local paths connects:
at least one of the nodes on the reference genome path to at least one of the nodes off the reference genome path, or
at least two of the nodes off the reference genome path.

19. The computer-readable medium of claim 14, wherein the one or more variants include at least one of:
an insertion into the graph-based reference genome;
a deletion in the graph-based reference genome; or
a replacement in the graph-based reference genome.

20. The computer-readable medium of claim 14, further comprising:
determining a pattern based on the one or more variants,
wherein the pattern corresponds to a propensity for a subject to contract a disease or guidelines for performing a clinical trial for drug approval.
